(19) United States
(12) Patent Application Publication
(10) Pub. No.: US 2008/0194811 A1
(43) Pub. Date: Aug. 14, 2008

(54) Catalysts and Methods for Complex Carbohydrate Hydrolysis

(76) Inventor: Larry C. McNeff, Anoka, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 12/030,649

(22) Filed: Feb. 13, 2008

Related U.S. Application Data
(60) Provisional application No. 60/889,730, filed on Feb. 13, 2007.
(60) Provisional application No. 60/911,313, filed on Apr. 12, 2007.

(51) Int. Cl. C07H 1/00; B01J 19/00

(52) U.S. Cl. 536/124; 422/212

(57) ABSTRACT

The present invention relates to methods and catalysts for hydrolyzing complex carbohydrates. In an embodiment, the invention includes a process for producing monosaccharides from a complex carbohydrate feedstock including the operations of heating the complex carbohydrate feedstock to a temperature greater than about 150 degrees Celsius and contacting the complex carbohydrate feedstock with a metal oxide catalyst. In an embodiment, the invention includes a method of hydrolyzing complex carbohydrates including the operations of heating the complex carbohydrate feedstock to a temperature greater than about 150 degrees Celsius and passing the complex carbohydrate feedstock through a housing to form a reaction product mixture. In an embodiment, the invention includes a polysaccharide hydrolysis reactor including a reactor housing and a catalyst disposed within the reactor housing. Other embodiments are also described herein.

This application claims the benefit of U.S. Provisional Application No. 60/889,730, filed Feb. 13, 2007, and U.S. Provisional Application No. 60/911,313, filed Apr. 12, 2007, the contents of all of which are herein incorporated by reference in their entirety.

### FIELD OF THE INVENTION

The present invention relates to methods and catalysts for breaking down carbohydrates. More specifically, the invention relates to methods and catalysts for hydrolyzing complex carbohydrates.

### BACKGROUND OF THE INVENTION

Carbohydrates are fundamentally important molecules to living organisms. Carbohydrates include a group of organic compounds based on the general formula Cₓ(H₂O)_{y}. The group specifically includes monosaccharides, disaccharides, oligosaccharides, polysaccharides (sometimes called “glycans”), and their derivatives. Some carbohydrates serve as a chemical store of energy for living organisms. For example, glucose is a monosaccharide found in fruits, honey, and the blood of many animals, that can be readily metabolized by many organisms to provide energy. Glucose also has many industrial uses, including as a feedstock for microbial ethanol production.

However, most complex carbohydrates are not as readily usable by living organisms as glucose. For example, cellulose and starch are polysaccharides that are primarily produced by plants as a structural component of their cell walls. These polysaccharides are largely insoluble in water and are not readily metabolized by most organisms without reduction to simpler sugars. However, polysaccharides are widely considered to be the most abundant organic compound in the biosphere. As such, the breakdown of polysaccharides, such as cellulose, into simple sugars has been the focus of significant research efforts.

Currently, there are two main approaches used to breakdown complex carbohydrates into more readily usable simple sugar molecules. The first approach is the acid mediated hydrolysis of complex carbohydrates. In this approach, a strong acid is combined with the complex carbohydrate at room temperature or at an elevated temperature and the complex carbohydrate is broken down into a mixture of components including monosaccharides and disaccharides. Unfortunately, strong acids are usually highly caustic and can create safety issues. In addition, recovery of the acid after the reaction makes this approach relatively costly and time consuming.

Another approach is the enzymatic hydrolysis of complex carbohydrates. In this approach, an enzyme is added to a mixture of complex carbohydrates resulting in hydrolytic cleavage and usually producing a mixture of monosaccharides and disaccharides. Unfortunately, these enzymatic reactions generally take a significant amount of time to reach completion. In addition, because the enzymes are proteins, they are subject to denaturation (wherein they lose their enzymatic capability) and are relatively fragile (chemically and thermally), constraining the possible reaction conditions. Finally, enzymes are relatively expensive to produce.

For at least these reasons, a need exists for new methods and catalysts for breaking down complex carbohydrates into useful chemical compounds.

### SUMMARY OF THE INVENTION

The present invention relates to methods and catalysts for hydrolyzing complex carbohydrates. In an embodiment, the invention includes a process for producing monosaccharides from a complex carbohydrate feedstock including the operations of heating the complex carbohydrate feedstock to a temperature greater than about 150 degrees Celsius and contacting the complex carbohydrate feedstock with a catalyst comprising a metal oxide selected from the group consisting of zirconia, alumina, hafnia and titania.

In an embodiment, the invention includes a method of hydrolyzing complex carbohydrates including the operations of heating the complex carbohydrate feedstock to a temperature greater than about 150 degrees Celsius and passing the complex carbohydrate feedstock through a housing to form a reaction product mixture, the housing including a catalyst. The catalyst including a metal oxide selected from the group consisting of zirconia, alumina, hafnia and titania.

In an embodiment, the invention includes a polysaccharide hydrolysis reactor including a reactor housing, the reactor housing defining an interior volume, a feedstock input port, and a reaction product output port. The reactor can also include a conveying mechanism disposed within the interior volume of the reactor housing configured to mix and move contents disposed within the interior volume of the reactor housing from the feedstock input port to the reaction product output port. The reactor can also include a catalyst disposed within the reactor housing, the catalyst comprising a metal oxide selected from the group consisting of zirconia, alumina, hafnia, and titania.

In an embodiment, the invention includes a polysaccharide extraction chamber configured for use with a supercritical fluid (such as water) that is conveyed by a high pressure pump which can then be passed to hydrolysis reactor including a reactor housing, the reactor housing defining an interior volume, a feedstock input port, and a reaction product output port. The reactor can also include a catalyst disposed within the reactor housing, the catalyst comprising a metal oxide selected from the group consisting of zirconia, alumina, hafnia, and titania. Multiple extraction chambers can be extracted in series to make the process semi-continuous and fully automated.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a schematic view of a complex carbohydrate reactor in accordance with an embodiment of the invention.

FIG. 2 is a schematic view of a complex carbohydrate reactor in accordance with another embodiment of the invention.

FIG. 3 is a schematic view of an extraction vessel in accordance with an embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The term “complex carbohydrate” as used herein shall refer to chemical compounds having two or more saccharide units. As such complex carbohydrates shall specifically include disaccharides, oligosaccharides, and polysaccharides.

As described above, carbohydrates can serve as a chemical store of energy. Unfortunately, this energy cannot be readily extracted from some carbohydrates, such as some complex carbohydrates. For example, cellulose and starch, complex carbohydrates, are widely considered to be the most abundant organic compounds in the biosphere, but they cannot directly be used by most organisms without breakdown to simpler carbohydrates. Embodiments of the invention include catalysts and methods for breaking down complex carbohydrates into more useful molecules. More specifically, embodiments of the invention relate to methods and catalysts for hydrolyzing complex carbohydrates. Hydrolysis of complex carbohydrates involves the cleavage of chemical bonds between adjacent saccharide units. As an example, the hydrolysis of both cellulose and starch are illustrated in the diagram below:

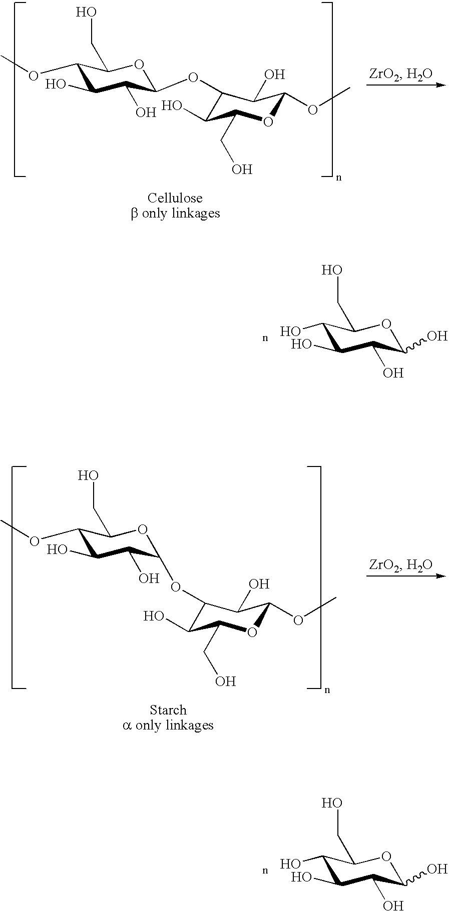

As described above, there are currently two main commercial approaches used to breakdown carbohydrates into more readily usable molecules. The first approach is the acid mediated hydrolysis of complex carbohydrates. In this approach, a strong acid, such as concentrated sulfuric acid, is combined with the complex carbohydrate leading to hydrolysis of the complex carbohydrate into a mixture of components including monosaccharides and disaccharides. Unfortunately, strong acids are usually highly caustic and can their use can create safety issues. In addition, recovery of the acid after the reaction makes this approach relatively costly and time consuming.

Another approach is the enzymatic hydrolysis of complex carbohydrates. In this approach, an enzyme is added to a mixture of complex carbohydrates resulting in hydrolytic cleavage and usually producing a mixture of monosaccharides and disaccharides. Unfortunately, these enzymatic reactions generally take a significant amount of time to reach completion. In addition, because the enzymes are proteins, they are subject to denaturation and relatively fragile, constraining the possible reaction conditions. Finally, enzymes are relatively expensive to produce.

However, as demonstrated herein, the hydrolysis of complex carbohydrates can be efficiently catalyzed by certain metal oxides. In an embodiment, the invention includes a process for producing monosaccharides from a complex carbohydrate feedstock including the operations of heating the complex carbohydrate feedstock to a temperature greater than about 150 degrees Celsius and passing the complex carbohydrate feedstock over a catalyst comprising a metal oxide selected from the group consisting of zirconia, alumina, hafnia and titania.

While not intending to be bound by theory, it is believed that the use of metal oxides to catalyze the hydrolysis of complex carbohydrates can offer various advantages. For example, metal oxide catalysts used with embodiments of the invention are extremely durable making them conducive to use in many different potential processing steps. In addition, such metal oxide catalysts can be reused many times, making this approach cost effective. Further, metal oxide catalysts used with embodiments of the invention do not create the same types of handling hazards created by the use of caustic acids, such as sulfuric acid.

Metal oxides catalysts used with embodiments of the invention can include metal oxides whose surfaces are dominated by Lewis acid-base chemistry. By definition, a Lewis acid is an electron pair acceptor. Metal oxides of the invention can have Lewis acid sites on their surface and can specifically include zirconia, alumina, titania and hafnia. Metal oxides of the invention can also include silica clad with a metal oxide selected from the group consisting of zirconia, alumina, titania, hafnia, zinc oxide, copper oxide, magnesium oxide and iron oxide. Metal oxides of the invention can also include mixtures of metal oxides specifically mixtures of zirconia, alumina, titania and/or hafnia. However, in other embodiments the metal oxide catalyst may include substantially pure zirconia, alumina, titania, and/or hafnia. Of the various metal oxides that can be used with embodiments of the invention, zirconia, titania and hafnia are advantageous as they are very chemically and thermally stable and can withstand very high temperatures and pressures as well as extremes in pH.

Metal oxides of the invention can include metal oxide particles clad with carbon. Carbon clad metal oxide particles can be made using various techniques such as the procedures described in U.S. Pat. Nos. 5,108,597, 5,254,262, 5,346,619, 5,271,833, and 5,182,016, the contents of which are herein incorporated by reference. Carbon cladding on metal oxide particles can render the surface of the particles more hydrophobic.

Metal oxides of the invention can also include polymer coated metal oxides. By way of example, metal oxides of the invention can include a metal oxide coated with polybutadiene (PBD). Polymer coated metal oxide particles can be made using various techniques such as the procedure described in Example 1 of U.S. Pub. Pat. App. No. 2005/0118409, the contents of which is herein incorporated by reference. Polymer coatings on metal oxide particles can render the surface of the particles more hydrophobic.

Metal oxide catalysts of the invention can be made in various ways. As one example, a colloidal dispersion of zirconium dioxide can be spray dried to produce aggregated zirconium dioxide particles. Colloidal dispersions of zirconium dioxide are commercially available from Nyacol Nano Technologies, Inc., Ashland, Mass. The average diameter of particles produced using a spray drying technique can be varied by changing the spray drying conditions. Examples of spray drying techniques are described in U.S. Pat. No. 4,138,336 and U.S. Pat. No. 5,108,597, the contents of both of which are herein incorporated by reference. It will be appreciated that other methods can also be used to create metal oxide particles. One example is an oil emulsion technique as described in Robichaud et al., Technical Note, “An Improved Oil Emulsion Synthesis Method for Large, Porous Zirconia Particles for Packed- or Fluidized-Bed Protein Chromatography,” Sep. Sci. Technol. 32, 2547-59 (1997). A second example is the formation of metal oxide particles by polymer induced colloidal aggregation as described in M. J. Annen, R. Kizhappali, P. W. Carr, and A. McCormick, “Development of Porous Zirconia Spheres by Polymerization-Induced Colloid Aggregation-Effect of Polymerization Rate,” J. Mater. Sci. 29, 6123-30 (1994). A polymer induced colloidal aggregation technique is also described in U.S. Pat. No. 5,540,834, the contents of which is herein incorporated by reference.

Metal oxide catalysts used in embodiments of the invention can be sintered by heating them in a furnace or other heating device at a relatively high temperature. In some embodiments, the metal oxide is sintered at a temperature of 160° C. or greater. In some embodiments, the metal oxide is sintered at a temperature of 400° C. or greater. In some embodiments, the metal oxide is sintered at a temperature of 600° C. or greater. Sintering can be done for various amounts of time depending on the desired effect. Sintering can make metal oxide catalysts more durable. In some embodiments, the metal oxide is sintered for more than about 30 minutes. In some embodiments, the metal oxide is sintered for more than about 3 hours. However, sintering also reduces the surface area. In some embodiments, the metal oxide is sintered for less than about 1 week.

In some embodiments, the metal oxide catalyst is in the form of particles. Particles within a desired size range can be specifically selected for use as a catalyst. For example, particles can be sorted by size such as by air classification, elutriation, settling fractionation, or mechanical screening. In some embodiments, the size of the particles is greater than about 0.2 μm. In some embodiments, the size range selected is from about 0.2 μm to about 1 mm. In some embodiments, the size range selected is from about 1 μm to about 100 μm. In some embodiments, the size range selected is from about 5 μm to about 15 μm. In some embodiments, the size range selected is about 10 μm. In some embodiments, the size range selected is about 5 μm.

In some embodiments, metal oxide particles used with embodiments of the invention are porous. By way of example, in some embodiments the metal oxide particles can have an average pore size of about 30 angstroms to about 2000 angstroms. However, in other embodiments, metal oxide particles used are non-porous.

The Lewis acid sites on metal oxides of the invention can interact with Lewis basic compounds. Thus, Lewis basic compounds can be bonded to the surface of metal oxides of the invention. A Lewis base is an electron pair donor. Lewis basic compounds of the invention can include anions formed from the dissociation of acids such as hydrobromic acid, hydrochloric acid, hydroiodic acid, nitric acid, sulfuric acid, perchloric acid, boric acid, chloric acid, phosphoric acid, pyrophosphoric acid, methanethiol, chromic acid, permanganic acid, phytic acid and ethylenediamine tetramethyl phosphonic acid (EDTPA). Lewis basic compounds of the invention can also include hydroxide ion as formed from the dissociation of bases such as sodium hydroxide, potassium hydroxide, lithium hydroxide and the like.

The anion of an acid can be bonded to a metal oxide of the invention by refluxing the metal oxide in an acid solution. By way of example, metal oxide particles can be refluxed in a solution of sulfuric acid. Alternatively, the anion formed from dissociation of a base, such as the hydroxide ion formed from dissociation of sodium hydroxide, can be bonded to a metal oxide by refluxing in a base solution. By way of example, metal oxide particles can be refluxed in a solution of sodium hydroxide. The base or acid modification can be achieved under exposure to the acid or base in either batch or continuous flow conditions when disposed in a reactor housing at elevated temperature and pressure to speed up the adsorption/modification process. In some embodiments, fluoride ion, such as formed by the dissociation of sodium fluoride, can be bonded to the particles.

In some embodiments, metal oxide particles can be packed into a housing, such as a column. Disposing metal oxide particles in a housing is one approach to facilitating continuous flow processes. Many different techniques may be used for packing the metal oxide particles into a housing. The specific technique used may depend on factors such as the average particle size, the type of housing used, etc. Generally speaking, particles with an average size of about 1-20 microns can be packed under pressure and particles with an average size larger than 20 microns can be packed by dry-packing/tapping methods or by low pressure slurry packing. In some embodiments, the metal oxide particles of the invention can be impregnated into a membrane, such as a PTFE membrane.

However, in some embodiments, metal oxide catalysts used with embodiments of the invention are not in particulate form. For example, a layer of a metal oxide can be disposed on a substrate in order to form a catalyst used with embodiments of the invention. The substrate can be a surface that is configured to contact the complex carbohydrate feed stock during processing. In one approach, a metal oxide catalyst can be disposed as a layer over a surface of a reactor that contacts the complex carbohydrate feed stock. Alternatively, the metal oxide catalyst can be embedded as a particulate in the surface of an element that is configured to contact the complex carbohydrate feed stock during processing.

In some embodiments, an additive can be added to the carbohydrate feed stock before or during processing. For example, water can be added to the complex carbohydrate feed stock before and/or during processing. The water can serve various purposes including helping to reduce the viscosity of the carbohydrate feedstock and facilitating the degree of completion of the hydrolysis reaction and increasing the contact between the catalyst and the complex carbohydrate.

As another example of an additive, a carrier compound can be added to the complex carbohydrate feed stock before or during processing. The carrier compound can be a compound that is non-reactive under the reaction conditions. Examples of carrier compounds can include, but are not limited to, hexane, saturated cycloalkanes, and fluorinated hydrocarbons. Carrier compounds can be present in the reaction mixture in an amount from 0.0 wt. % to 99.9 wt. %. Conversely, active components, such as the lipid feedstock and the alcohol feedstock can be present in the reaction mixture in an amount from 0.1 wt. % to 100.0 wt. %.

As demonstrated below in Example 4, the hydrolysis of a complex carbohydrate using a metal oxide catalyst is temperature dependent. If the temperature is not high enough, the hydrolysis reaction will not proceed optimally. As such, in some embodiments, the complex carbohydrate feedstock is heated to about 1500 Celsius or hotter. In some embodiments, the complex carbohydrate feedstock is heated to about 200° Celsius or higher.

However, while not intending to be bound by theory, it is believed that if the temperature of the reaction is too high, the reaction products will consist of significant portions of gases, such as carbon dioxide and hydrogen, because monosaccharides will break down into these elementary components in the presence of metal oxide catalysts at high temperatures. As such, if the desired end product is a monosaccharide, it can be advantageous to limit the temperature of the reaction. In some embodiments, the complex carbohydrate feedstock is kept at a temperature of less than about 300° Celsius. In some embodiments, the complex carbohydrate feedstock is kept at a temperature of less than about 250° Celsius. In some embodiments, the complex carbohydrate feedstock is heated to a temperature of between about 150° Celsius and about 250° Celsius. In some embodiments, the complex carbohydrate feedstock is heated to a temperature of between about 1800 Celsius and about 220° Celsius.

While not intending to be bound by theory, it is believed that the desired end product can also be controlled by modulating the contact time. In an embodiment, the contact time is between about 0.1 seconds and 2 hours. In an embodiment, the contact time is between about 1 second and 20 minutes. In an embodiment, the contact time is between about 2 seconds and 1 minute.

### Complex Carbohydrate Hydrolysis Reactors

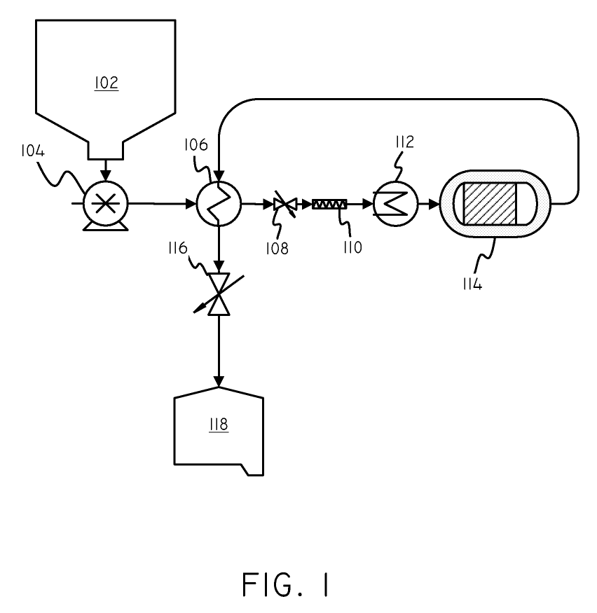

It will be appreciated that many different reactor designs are possible in order to perform methods and processes as described herein. Specific design choices can be influenced by various factors including, significantly, the nature of the complex carbohydrate feed stock. In some cases, the complex carbohydrate feedstock may be substantially liquefied because of a significant amount of water or another solvent. Referring now to FIG. 1, a schematic diagram is shown of a complex carbohydrate hydrolysis reactor in accordance with an embodiment of the invention suitable for use with substantially liquefied feedstocks. In this embodiment, a complex carbohydrate feedstock is held in a tank 102. In some embodiments, the tank 102 can be heated.

The complex carbohydrate feedstock then passes through a pump 104 before passing through a heat exchanger 106 where the feedstock absorbs heat from downstream products. An exemplary counter-flow heat exchanger is described in U.S. Pat. No. 6,666,074, the contents of which are herein incorporated by reference. For example, a pipe or tube containing the effluent flow is routed past a pipe or tube holding the feed stock flow or the reaction mixture. In some embodiments, a thermally conductive material, such as a metal, connects the effluent flow with the feedstock flow so that heat can be efficiently transferred from the effluent products to the incoming feedstock. Transferring heat from the effluent flow to the feedstock flow can make the production process more energy efficient since less energy is used to get the reaction mixture up to the desired temperature.

The complex carbohydrate feedstock tank may be continuously sparged with an inert gas such as nitrogen to remove dissolved oxygen from the feedstock. The complex carbohydrate feedstock passes through a shutoff valve 108 and, optionally, a filter 110 to remove particulate material of a certain size from the feedstock stream. The complex carbohydrate feedstock then passes through a preheater 112. The preheater 112 can elevate the temperature of the reaction mixture to a desired level. Many different types of heaters are known in the art and can be used.

The reaction mixture can then pass through a reactor 114 where the complex carbohydrate feedstock is converted into a reaction product mixture including monosaccharides. The reactor can include a metal oxide catalyst, such as in the various forms described herein. In some embodiments the reactor housing is a ceramic that can withstand elevated temperatures and pressures. In some embodiments, the reactor housing is a metal or an alloy of metals. Next, the reaction product mixture can pass through the heat exchanger 106 in order to transfer heat from the effluent reaction product stream to the complex carbohydrate feedstock stream. The reaction product mixture can also pass through a backpressure regulator 116 before passing on to a reaction product storage tank 118.

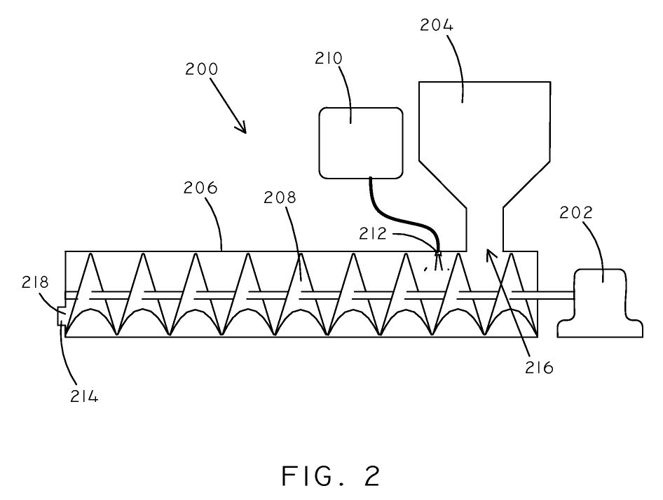

In some embodiments, the complex carbohydrate feedstock stream may not be in a substantially liquefied state. Referring now to FIG. 2, a schematic diagram is shown of a complex carbohydrate hydrolysis reactor 200 in accordance with an embodiment of the invention suitable for use with substantially non-liquefied feedstocks. The reactor 200 includes a reactor housing 206 defining an input port 216 and an output port 218. A hopper 204 is configured to hold a solid or semi-solid complex carbohydrate feedstock and deliver it into the reactor housing 206 through the input port 216. The complex carbohydrate feedstock is conveyed and mixed by an extrusion screw 208. The extrusion screw 208 is rotated by a motor 202.

Various additives can be inserted into the reactor housing 206. For example, additives can be stored in an additive tank 210 and then injected into the reactor housing 206 through an additive injection port 212. Additives can include metal oxide catalysts, water, surfactants, acids or bases, carrier compounds, scent precursors or the like.

In some embodiments, a temperature control system (not shown) can be disposed along the reactor housing 206 in order to maintain the interior of the reactor housing at a given temperature. In some embodiments, a preheater (not shown) can be disposed along the hopper 204 in order to heat the complex carbohydrate feedstock to a desired temperature before it enters the reactor housing 206.

The reactor 200 is configured to allow the complex carbohydrate feedstock stream to interact with a metal oxide catalyst. In some embodiments, a metal oxide catalyst can be embedded in the walls of the reactor housing 206. In some embodiments, a metal oxide catalyst can be embedded on the surfaces of the extrusion screw 208. In some embodiments, a particulate metal oxide catalyst is added to the complex carbohydrate feedstock before entering the reactor housing 206 and then later recovered after passing through the reactor housing 206.

The extrusion screw 208 rotates and moves the complex carbohydrate feedstock through the reactor housing 206 toward the output port 218. Pressure and, as a result, temperature are increased as the complex carbohydrate feedstock is pushed on by the extrusion screw 208. The elevated temperature within the reactor housing 206, in combination with exposure to the metal oxide catalyst, hydrolyzes the carbohydrate feedstock stream into a reaction product stream containing monosaccharides. The reaction product stream passes out of the reactor housing 206 and then through an extrusion die 214.

Though not shown in FIGS. 1-2, in some embodiments, complex carbohydrate feedstocks can be subjected to one or more preprocessing steps before being processed in a reactor. For example, a complex carbohydrate feedstock can be subject to mechanical processing in order to render the complex carbohydrates therein more suitable for reaction. In some embodiments, the complex carbohydrate feedstock may be mechanically processed to yield a relatively fine particulate feedstock. By way of example, mechanical processing can include operations of cutting, chopping, crushing, grinding, or the like. In some embodiments, other types of processing procedures can be performed such as the addition of water, or other additives, to the complex carbohydrate feedstock.

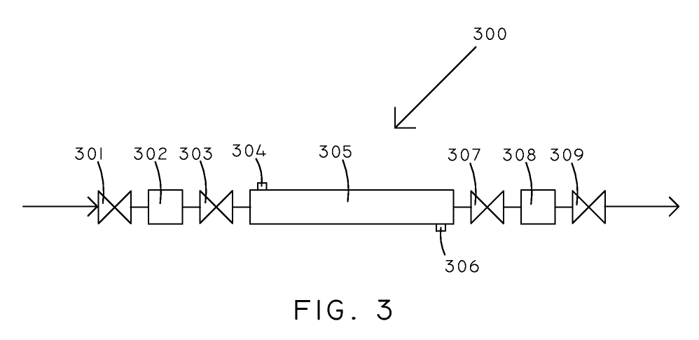

In some embodiments, a feedstock may be subjected to an extraction operation before contacting a metal oxide catalyst. For example, a complex carbohydrate feedstock can be subjected to a supercritical fluid extraction operation. One example of a supercritical fluid extraction apparatus is described in U.S. Pat. No. 4,911,941, the contents of which is herein incorporated by reference. Referring now to FIG. 3, a complex carbohydrate extraction system 300 is shown in accordance with an embodiment of the invention. At steady state conditions, the extraction vessel 305 is filled with a raw feedstock material that contains complex carbohydrates. A supercritical fluid is fed to the first end 306 of the extraction vessel 305 and complex carbohydrate-containing supercritical fluid is withdrawn from the second end 304 of the extraction vessel 305. In an embodiment, the supercritical fluid is supercritical water. In an embodiment, the supercritical fluid is carbon dioxide. Raw feedstock material is periodically admitted through valve 301 into blow case 302. Valves 303 and 307 are simultaneously opened intermittently so as to charge the raw feedstock from blow case 302 to the second end of the extraction vessel 304 and discharge a portion of processed feedstock waste from the first end 306 of the extraction vessel 305 to blow case 308. Valves 303 and 307 are then closed. Valve 309 is then opened to discharge the processed feedstock waste from blow case 308. Additional raw feedstock is admitted through valve 301 into blow case 302 and the procedure is repeated. The extraction system 300 can be connected in series with a complex carbohydrate reactor. For example, the extraction system 300 can be connected in series with the complex carbohydrate reactor shown in FIG. 2.

In some embodiments, the complex carbohydrate feedstock is kept under pressure during the reaction in order to prevent components of the reaction mixture (the complex carbohydrate feedstock and any additives) from vaporizing. The reactor housing can be configured to withstand the pressure under which the reaction mixture is kept. A desirable pressure for the reactor can be estimated with the aid of the Clausius-Clapeyron equation. Specifically, the Clausius-Clapeyron equation can be used to estimate the vapor pressures of a liquid. The Clausius-Clapeyron equation is as follows:

$$ln ( \frac{{P}_{1}}{{P}_{2}} ) = \frac{Δ {H}_{vap}}{R} ( \frac{1}{{T}_{2}} - \frac{1}{{T}_{1}} )$$

wherein ΔHᵥₐₚ=is the enthalpy of vaporization; P₁ is the vapor pressure of a liquid at temperature T₁; P₂ is the vapor pressure of a liquid at temperature T₂, and R is the ideal gas law constant.

In an embodiment, the pressure inside the housing is greater than the vapor pressures of any of the components of the reaction mixture. In an embodiment, the pressure is greater than about 500 psi. In an embodiment, the pressure is greater than about 800 psi. In an embodiment, the pressure is greater than about 1000 psi. In an embodiment, the pressure is greater than about 1500 psi. In an embodiment, the pressure is greater than about 2000 psi. In an embodiment, the pressure is greater than about 3000 psi. In an embodiment, the pressure is greater than about 3000 psi. In an embodiment, the pressure is greater than about 4000 psi. In an embodiment, the pressure is greater than about 5000 psi.

The reaction mixture may be passed over the metal oxide catalyst for a length of time sufficient for the reaction to reach a desired level of completion. This will in turn depend on various factors including the temperature of the reaction, the chemical nature of the catalyst, the surface area of the catalyst, the contact time with the catalyst and the like.

In some embodiments, the reaction mixture reaches the desired level of completion after one pass over the metal oxide catalyst bed or packing. However, in some embodiments, the effluent flow may be rerouted over the same metal oxide catalyst or routed over another metal oxide catalyst bed or packing so that reaction is pushed farther toward completion in stages.

In some embodiments two or more metal oxide catalyst beds can be used to convert complex carbohydrate feedstocks to monosaccharide containing products. In some embodiments, an acid-modified metal oxide catalyst (such as sulfuric or phosphoric acid modified) and a base-modified metal oxide catalyst (such as sodium hydroxide modified) can be separately formed but then disposed together within a single reactor housing. In such an approach, the reaction mixture passing through the reactor housing can be simultaneously exposed to both the acid and base modified metal oxide catalysts.

In some embodiments, two different metal oxides (such zirconia and titania) can be separately formed but then disposed together within a single reactor housing. In such an approach, the reaction mixture passing through the reactor housing can be simultaneously exposed to both metal oxide catalysts.

In some embodiments, one or more metal oxides (such zirconia and titania) can be coated on an inert porous support (such as silica gel) separately formed but then disposed together within a single reactor housing. In such an approach, the reaction mixture passing through the reactor housing can be simultaneously exposed to the metal oxide catalyst(s).

### Complex Carbohydrate Feed Stocks

As complex carbohydrates are a significant component of biomass, it will be appreciated that complex carbohydrates feedstocks useful with embodiments of the invention can be derived from elements of many different plants, animals, microbes, and other living organisms. Virtually any living organism is a potential source of biomass for use as a complex carbohydrate feed stock. Complex carbohydrate feedstocks can be derived from industrial processing wastes, food processing wastes, mill wastes, municipal/urban wastes, forestry products and forestry wastes, agricultural products and agricultural wastes, amongst other sources. Complex carbohydrates found in these sources can include cellulose, hemicellulose, agar, guar gum, starch, and xylan, amongst other carbohydrates. In some embodiments, the complex carbohydrate feed stock can include at least about 10 wt. % cellulose. In some embodiments, the complex carbohydrate feed stock can include at least about 10 wt. % starch.

Though not limiting the scope of possible sources, specific examples of biomass crop sources can include poplar, switchgrass, reed canary grass, willow, silver maple, black locust, sycamore, sweetgum, sorghum, miscanthus, eucalyptus, hemp, maize, wheat, soybeans, alfalfa, and prairie grasses.

The present invention may be better understood with reference to the following examples. These examples are intended to be representative of specific embodiments of the invention, and are not intended as limiting the scope of the invention.

### EXAMPLES

### Example 1

### Formation of Zirconia Particles

A colloidal dispersion of zirconium oxide (NYACOL™ ZR 100/20) (Nyacol Nano Technologies, Inc., Ashland, Mass.), containing 20 wt. % ZrO₂ primarily as about 100 nm particles was spray dried. As the dispersion dried, the particles interacted strongly with one another to provide aggregated ZrO₂ particles. The dried aggregated particles that were obtained were examined under an optical microscope and observed to consist mostly of spherules from about 0.5 μm to about 15 μm in diameter.

The dried spherules were then sintered by heating them in a furnace at a temperature of 750° C. for 6 hours. The spherules were air classified, and the fraction having a size of approximately 10 μm was subsequently isolated. The particles were all washed in sodium hydroxide (1.0 Molar), followed by water, nitric acid (1.0 Molar), water and then dried under vacuum at 110° C. BET nitrogen porosimetry was performed in order to further characterize the sintered spherules. The physical characteristics of the spherules were as listed below in Table 1.

TABLE 1

| | |
|---|---|
| Surface area (m\hat{ }2/g) | 22.1 |
| Pore volume (mL/g) | 0.13 |
| Pore diameter (angstrom) | 240 |
| Internal Porosity | 0.44 |
| Average size range (micron) | 5-15 |
| Size Standard Deviation (um) | 2.62 |
| D90/D10 (Size Distribution) | 1.82 |

### Example 2

### Formation of Base Modified Zirconia Particles

1 liter of 2.0 M sodium hydroxide was placed in a 2 liter plastic Erlenmeyer flask. 110 g of 5-15 μm bare zirconia prepared as described in Example 1 was put into the flask. The particle suspension was sonicated for 10 minutes under vacuum and then swirled for 2 hours at ambient temperature. The particles were then allowed to settle and the alkaline solution was decanted and then 1.4 liters of HPLC-grade water was added to the flask followed by settling and decanting. Then 200 mL of HPLC-grade water was added back to the flask and the particles were collected on a nylon filter with 0.45 micron pores. The collected particles were then washed with 2 aliquots of 200 mL HPLC-grade water followed by 3 aliquots of 200 mL of HPLC-grade methanol. Air was then allowed to pass through the particles until they were free-flowing.

### Example 3

### Formation of a Packed Column

Particles as formed in Example 3 were slurried in methanol (26 g zirconia in 44 mL of methanol) and packed into a 15 cm×10.0 mm i.d. stainless steel HPLC column at 7,000 PSI using methanol as a pusher solvent. The column was allowed to pack for 8 minutes under pressure and then the pressure was allowed to slowly bleed off and the end fitting and frit were attached to the inlet of the column. 200 mL of total solvent was collected in the packing process.

### Example 4

### Conversion of Starch to Glucose

A starch feedstock was prepared to serve as an example of a complex carbohydrate feedstock. Specifically, 1523.80 g HPLC water was put into a 2000 mL beaker. 50 grams of starch (Sigma-Aldrich Catalog No.: 33615, starch soluble puriss, (Riedel-deHaen), Lot no.: 6191A) was added to the beaker. The contents were heated to 70° C. to dissolve the starch. The resulting solution was observed to be milky. The solution was centrifuged at 3750 rpm for 10 minutes. The remaining solution was decanted and filtered with a 0.45 micron NYLON HPLC solvent filter (Millipore).

Next, a reactor apparatus was setup. The reactor apparatus including a flask disposed on a hot plate to store the starch feedstock solution and an Omega temperature controller to monitoring the temperature of the feedstock solution. A feed stock supply line (stainless steel tubing) connected the flask with an HPLC pump (Waters 590), passing through a resistive preheater. The resistive preheater was formed by wrapping the tubing in a groove around an aluminum block with a Watlow heater in the center of the block. An OMEGA controller measures and controls the temperature of the preheater. The feedstock solution was sparged continuously with nitrogen to displace dissolved oxygen. The HPLC pump was, in turn, connected to two 10 mm i.d.×15 cm columns (in series) packed with base modified zirconia, prepared as described in example 2 above. The temperature of the columns was regulated using a column heating apparatus (resistive Watlow tube furnace heater connected to a Variac to control the amount of current flow and therefore the temperature). After passing through the columns, the reaction product mixture passed through a back pressure regulator and a heat exchanger.

Next, samples of the starch feedstock were processed through the reaction apparatus under varying conditions. Specifically, the reaction was carried out under the conditions described below in Table 2.

TABLE 2

| | Temperatures (° C.) | | | | | |
|---|---|---|---|---|---|---|
| | | | | 2nd | Pressures | |
| | | 1st Column | Between | Column | (PSI) | |
| Sample | Preheater | Inlet | Columns | Outlet | Front | Back |
| 1 | 163 | 161 | 158 | 157 | 2500 | 2100 |
| 2 | 164 | 163 | 160 | 160 | 2600 | 2350 |
| 3 | 211 | 197 | 192 | 188 | 2500 | 2300 |
| 4 | 208 | 202 | 195 | 193 | 3300 | 3000 |
| 5 | 212 | 205 | 197 | 195 | 2800 | 2600 |
| 6 | 211 | 205 | 197 | 195 | 3150 | 3050 |
| 7 | 206 | 198 | 197 | 195 | 2700 | 2500 |
| 8 | 211 | 204 | 197 | 196 | 2700 | 2500 |
| 9 | 263 | 238 | 220 | 208 | 3200 | 2900 |

Glucose concentrations in the reaction product were assessed using a One Touch Ultra 2 blood glucose meter with a stated detection range of 20-600 mg/deciliter (commercially available from Johnson & Johnson). The results are shown in Table 3 below.

TABLE 3

| | | Flow | Glucose | |
|---|---|---|---|---|
| | Residence | Rate | Concentration | % |
| Sample | Time | (mL/min) | (mg/ml) | Conversion |
| 1 | 3.02 | 5.3 | Not Detected | 0 |
| 2 | 3.02 | 5.3 | Not Detected | 0 |
| 3 | 3.02 | 5.3 | 17.2 | 52 |
| 4 | 3.02 | 5.3 | 27.5 | 83 |
| 5 | 3.02 | 5.3 | 31 | 93 |
| 6 | 3.02 | 5.3 | 29.7 | 89 |
| 7 | 3.02 | 5.3 | 33.4 | 100 |
| 8 | 3.02 | 5.3 | 45.3 | 136* |
| 9 | 3.02 | 5.3 | 26 | 78 |

*value attributed to experimental error

The data show that a polysaccharide feedstock can be converted into a monosaccharide containing product using a metal oxide catalyst. The data further show a temperature dependence of the metal oxide catalyzed hydrolysis reaction. At the highest temperature (sample #9), gas formation was noted suggesting that at least part of the polysaccharide feedstock was transformed into carbon dioxide gas and hydrogen gas, reducing the yield of glucose.

The invention has been described with reference to various specific and preferred embodiments and techniques. However, it should be understood that many variations and modifications may be made while remaining within the spirit and scope of the invention.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

1. A process for producing monosaccharides from a complex carbohydrate feedstock comprising:
heating the complex carbohydrate feedstock to a temperature greater than about 150 degrees Celsius; and
contacting the complex carbohydrate feedstock with a catalyst comprising a metal oxide selected from the group consisting of zirconia, alumina, hafnia and titania.

2. The process of claim 1, the metal oxide comprising zirconia.

3. The process of claim 1, further comprising adding water to the complex carbohydrate feedstock.

4. The process of claim 1, comprising heating a complex carbohydrate feedstock to a temperature of between about 150 degrees Celsius and about 250 degrees Celsius.

5. The process of claim 1, further comprising subjecting the complex carbohydrate feed stock to a pressure greater than about 200 psi.

6. The process of claim 1, the complex carbohydrate feedstock comprising a component selected from the group consisting of starch and cellulose.

7. The process of claim 1, the complex carbohydrate feedstock comprising a material selected from the group consisting of wood chips, saw dust, cellulose fiber.

8. The process of claim 1, the catalyst comprising a particulate metal oxide, the particulate metal oxide comprising an average particle size of about 0.2 microns to about 1 millimeter.

9. The process of claim 1, the catalyst comprising a porous metal oxide.

10. A method of hydrolyzing complex carbohydrates comprising:
heating a complex carbohydrate feedstock to a temperature greater than about 150 degrees Celsius; and
passing the complex carbohydrate feedstock through a housing to form a reaction product mixture, the housing comprising a catalyst comprising a metal oxide selected from the group consisting of zirconia, alumina, hafnia and titania.

11. The method of claim 10, further comprising extruding the reaction product mixture out of an orifice.

12. The method of claim 10, comprising heating a complex carbohydrate feedstock to a temperature of between about 150 degrees Celsius and about 250 degrees Celsius.

13. The method of claim 10, further comprising subjecting the complex carbohydrate feed stock to a pressure greater than about 200 psi.

14. The method of claim 10, the complex carbohydrate feedstock comprising a component selected from the group consisting of starch and cellulose.

15. The method of claim 10, the catalyst comprising a porous metal oxide.

16. A polysaccharide hydrolysis reactor comprising:
a reactor housing, the reactor housing defining an interior volume, a feedstock input port, and a reaction product output port;
a conveying mechanism disposed within the interior volume of the reactor housing configured to mix and move contents disposed within the interior volume of the reactor housing from the feedstock input port to the reaction product output port; and
a catalyst disposed within the catalyst housing, the catalyst comprising a metal oxide selected from the group consisting of zirconia, alumina, hafnia, and titania.

17. The polysaccharide hydrolysis reactor of claim 16, the reactor housing comprising a housing wall surrounding the interior volume, the catalyst coupled to the housing wall.

18. The polysaccharide hydrolysis reactor of claim 16, further comprising a temperature controlled feedstock reservoir.

19. The polysaccharide hydrolysis reactor of claim 16, the reactor housing further defining a water injection port.

20. The polysaccharide hydrolysis reactor of claim 16, further comprising an extrusion die in fluid communication with the reaction product output port.

21. The polysaccharide hydrolysis reactor of claim 16, the conveying mechanism comprising an extrusion screw.

22. The polysaccharide hydrolysis reactor of claim 16, further comprising an extraction chamber in fluid communication with the reactor housing.
